(19) United States
(12) Patent Application Publication
(10) Pub. No.: US 2008/0200975 A1
(43) Pub. Date: Aug. 21, 2008

(54) Vascular Prosthesis with Anastomotic Member

(75) Inventor: Alexander Dubson, Petach-Tikva (IL)

Correspondence Address: Martin D. Moynihan;PRTSI, Inc., P.O. Box 16446, Arlington, VA, 22215, US

(73) Assignee: Nicast Ltd., Lod (IL)

(21) Appl. No.: 10/585,374

(22) PCT Filed: Jan. 4, 2005
(86) PCT No.: PCT/IL05/00011

§ 371 Date: Jul. 6, 2006

Related U.S. Application Data
(60) Provisional application No. 60/534,358, filed on Jan. 6, 2004.

(51) Int. Cl. A61F 2/06

(52) U.S. Cl. 623/1.15; 623/1.36; 623/1.44; 623/1.51

(57) ABSTRACT

A medical device for implantation in a vessel is disclosed. The medical device comprises at least one anastomotic member, at least partially interposing a non-woven liner of electrospun fibers and a non-woven cover of electrospun fibers. The at least one anastomotic member is designed for engaging at least one end of the medical device to a wall of the vessel upon implantation of the medical device within the vessel.

### FIELD AND BACKGROUND OF THE INVENTION

The present invention relates to implantable devices, and, more particularly, to a vascular prosthesis with anastomotic device.

Diseases affecting the vascular system are either congenital or acquired. An acquired vascular disease can result from living habits, infections or injuries during embryonic life, or at any time following birth. Some diseases primarily affect the blood vessels; others only the heart itself.

Atherosclerosis is the major disease that affects the blood vessels. This disease may have its beginnings early in life and is first noted as a thickening of the arterial walls. This thickening is an accumulation of fat, fibrin, cellular debris and calcium. The resultant narrowing of the internal lumen of the vessel is called stenosis. Vessel stenosis impedes and reduces blood flow. Hypertension and dysfunction of the organ or area of the body that suffered the impaired blood flow can result. As the buildup on the inner wall of a vessel thickens, the vessel wall loses the ability to expand and contract. Additionally, the vessel loses its viability and becomes weakened and susceptible to bulging, also known as aneurysm. In the presence of hypertension or elevated blood pressure, aneurysms frequently dissect and ultimately rupture.

Small vessels, such as the arteries that supply blood to the heart, legs, intestines and other areas of the body, are particularly susceptible to atherosclerotic narrowing. The loss of blood supply to the leg or segment of the intestine may result in gangrene. Atherosclerotic narrowing of the coronary arteries impedes limits and in some instances prevents blood flow to regional areas of the heart. Depending upon its severity and location within the coronary circulation, pain, cardiac dysfunction or death may result.

Vascular complications produced by atherosclerosis, such as, stenosis, aneurysm, rupture and occlusion are, in the majority of cases, managed either medically or surgically. Control and elimination of hypertension is the more effective form of medical management. Typically, surgical intervention is instituted in cases in which the atherosclerotic disease is advanced and the attendant complications jeopardize the health of the patient.

Aneurysms and stenosis of major arteries are best corrected by a plastic reconstruction that does not require any synthetic graft or patch materials. However, if the disease is extensive and the vessel is no longer reliable, the common practice is to replace the vessel or a portion thereof by a graft. In such cases, the involved vessel section is transected and removed and an artificial blood vessels is sewn into place. Another common practice for treating extensive stenosis, as well as other coronary heart diseases, is stent deployment. A stent is a generally cylindrical prosthesis introduced via a catheter into a lumen of a body vessel in a configuration having a generally reduced diameter and then expanded to the diameter of the vessel. The stent may be self-expanding, or it may be expandable by means of an inflatable portion of the catheter, such as a balloon. In its expanded configuration, the stent supports and reinforces the vessel walls while maintaining the vessel in an open, unobstructed condition.

Artificial blood vessels, vascular prostheses and stents, including self-expanding, inflation assisted expandable and inflation expandable stents, are well known and widely available in a variety of designs and configurations. Inflation expandable and inflation assisted expandable stents are expanded via outward radial pressure such as that provided by a balloon disposed underneath the stent during inflation of the balloon.

Of particular interest are prosthetic devices made of, or coated with, polymer materials which typically exhibit a microporous structure that in general allows healthy tissue growth and cell endothelization, thus contributing to the long term healing of the prostheses. Prostheses having sufficient porous structure tend to promote tissue ingrowth and cell endothelization along their inner surface.

Stents with fiber polymer coating promote preparation of porous coatings with better grafting and highly developed surface. For example, U.S. Pat. No. 5,549,663 discloses a stent graft having a coating made of polyurethane fibers which are applied using conventional wet spinning techniques. Prior to the covering process, a medication is introduced into the polymer.

A more promising method for stent coating is electrospinning. Electrospinning is a method for the manufacture of ultra-thin synthetic fibers which reduces the number of technological operations required in the manufacturing process and improves the product being manufactured in more than one way.

The process of electrospinning creates a fine stream or jet of liquid that upon proper evaporation of a solvent or liquid to solid transition state yields a nonwoven structure. The fine stream of liquid is produced by pulling a small amount of polymer solution through space by using electrical forces. More particularly, the electrospinning process involves the subjection of a liquefied substance, such as polymer, into an electric field, whereby the liquid is caused to produce fibers that are drawn by electric forces to an electrode, and are, in addition, subjected to a hardening procedure. In the case of liquid which is normally solid at room temperature, the hardening procedure may be mere cooling; however other procedures such as chemical hardening (polymerization) or evaporation of solvent may also be employed. The produced fibers are collected on a suitably located precipitation device and subsequently stripped from it. The sedimentation device is typically shaped in accordance with the desired geometry of the final product, which may be for example tubular, flat or even an arbitrarily shaped product.

The use of electrospinning for stent coating permits to obtain durable coating with wide range of fiber thickness (from tens of nanometers to tens of micrometers), achieves exceptional homogeneity, smoothness and desired porosity distribution along the coating thickness. Stents themselves do not encourage normal cellular invasion and therefore can lead to an undisciplined development of cells in the metal mesh of the stent, giving rise to cellular hyperplasia. When a stent is electrospinningly coated by a graft of a porous structure, the pores of the graft component are invaded by cellular tissues from the region of the artery surrounding the stent. Moreover, diversified polymers with various biochemical and physico-mechanical properties can be used in stent coating. Examples of electrospinning methods in stent manufacturing are found in U.S. Pat. Nos. 5,639,278, 5,723,004, 5,948,018, 5,632,772 and 5,855,598.

As the stent expands within the blood vessel, it then cracks the plaques on the wall of the artery and produces shards or fragments whose sharp edges cut into the tissue. This causes internal bleeding and a possible local infection, which if not adequately treated, may spread and adversely affect other parts of the body. Additionally, excessive natural healing process in response to arterial injuries, oftentimes results in re-closure (termed restenosis) of the treated artery. The natural healing process may continue until a complete reclusion of the artery.

The risk of vessel damage during stent implantation may be lowered through the use of a soft stent serving to improve the biological interface between the stent and the artery and thereby reduce the risk that the stent will inflict damage during implantation. Examples of polymeric stents or stent coatings with biocompatible fibers are found in, for example, U.S. Pat. Nos. 6,001,125, 5,376,117 and 5,628,788, all of which are hereby incorporated by reference.

U.S. Pat. No. 5,948,018 discloses a graft composed of an expansible stent component covered by an elastomeric polymeric graft component which, because of its stretchability, does not inhibit expansion of the stent. The graft component is fabricated by electrospinning to achieve porosity hence to facilitate normal cellular growth.

The U.S. Pat. No. 5,628,788 describes endoluminal self-expanding stent-graft with elastic jacket made of textile polyethylene terephthalate mesh. Such graft is able to repeat the stent shape and sizes after its expansion, thus providing easy connection with the operated vessel, on the one hand, and graft support within the entire life period, on the other hand. To achieve required sealing and prevent blood leakage, the stent-graft is supplemented by a porous elastomeric liner affixed to the mesh with an elastomeric adhesive. The liner increases the stent-graft as-folded diameter. Similar solutions were used in U.S. Pat. Nos. 5,632,772, 5,723,004, 5,855,598 and 5,948,018.

Even though coronary artery bypass surgery and other blood vessel implantation procedures are widely practiced and have become a routine procedure in hospitals throughout the world, they are not without certain operative limitations that would best be avoided. The procedure of stapling the graft to the host vessel, commonly termed anastomosis, is typically performed by suturing which may result in less than optimal result. Moreover, prior art vascular grafts are inapplicable for the treatment of, for example, complicated aneurysms such as abdominal, common iliac, external iliac, rental or mesenteric arteries, in which four or five arteries must be supported simultaneously.

There is thus a widely recognized need for, and it would be highly advantageous to have a vascular prosthesis with anastomotic device, devoid of the above limitations.

### SUMMARY OF THE INVENTION

According to one aspect of the present invention there is provided a medical device for implantation in a vessel, comprising at least one anastomotic member at least partially interposing a non-woven liner of electrospun fibers and a non-woven cover of electrospun fibers; the at least one anastomotic member being designed for engaging at least one end of the medical device to a wall of the vessel upon implantation of the medical device within the vessel.

According to another aspect of the present invention there is provided a kit for performing an end-to-side anastomosis procedure, comprising: a medical device for implantation in a vessel as described herein; and a accessory device for forming an opening in the wall of the vessel, the accessory device comprising a tubular encapsulation designed and constructed for receiving the medical device, a cutting member integrated with or attached to an end of the tubular encapsulation and capable of forming an opening in the wall of the vessel, and a vacuum channel for channeling efflux of biological material from the tubular encapsulation.

According to further features in preferred embodiments of the invention described below, the medical device forms a furcating structure having a plurality of tubular branches.

According to still further features in the described preferred embodiments the non-woven liner and the non-woven cover form a tubular structure.

According to still further features in the described preferred embodiments the at least one anastomotic member is adapted for end-to-end anastomosis.

According to still further features in the described preferred embodiments at least one end of the furcating structure is adapted for end-to-end anastomosis.

According to still further features in the described preferred embodiments at least one end of the tubular structure is adapted for end-to-end anastomosis.

According to still further features in the described preferred embodiments at least one anastomotic member is adapted for end-to-side anastomosis.

According to still further features in the described preferred embodiments at least one end of the furcating structure is adapted for end-to-side anastomosis.

According to still further features in the described preferred embodiments at least one end of the tubular structure is adapted for end-to-side anastomosis.

According to still further features in the described preferred embodiments the at least one anastomotic member protrudes out of the non-woven cover.

According to still further features in the described preferred embodiments the at least one anastomotic member comprises a single anastomotic member extending over the tubular structure and protruding from at least one end of the tubular structure.

According to still further features in the described preferred embodiments the at least one anastomotic member comprises a first anastomotic member engaging a first end of the tubular structure, and a second anastomotic member engaging a second end of the tubular structure.

According to still further features in the described preferred embodiments the at least one anastomotic member is flush with the non-woven cover.

According to still further features in the described preferred embodiments the at least one anastomotic member comprises an expandable supporting element. According to still further features in the described preferred embodiments the engagement of the at least one end of the medical device to the wall of the vessel is effected by pressure generated by an expansion of the expandable supporting element.

According to still further features in the described preferred embodiments the medical device further comprises a ring-shaped protuberance designed and constructed to prevent the device from falling into a lumen of the vessel.

According to still further features in the described preferred embodiments the at least one anastomotic member comprises a plurality of hooks for connecting the device to the wall of the vessel.

According to still further features in the described preferred embodiments the plurality of hooks is designed and constructed such that when the expandable supporting element expands, the plurality of hooks pierce the wall of the vessel, thereby mounting the device to the vessel.

According to still further features in the described preferred embodiments the plurality of hooks is designed and constructed to pierce an inner wall of the vessel and to protrude out of an outer wall of the vessel.

According to still further features in the described preferred embodiments the medical device further comprises at least one perforated member mounted on the non-woven cover in a manner such that protruding portions of the plurality of hooks engage the at least one perforated member.

According to still further features in the described preferred embodiments the medical device further comprises a pressing ring for bending protruding portions of the plurality of hooks, thereby perverting detachment of the plurality of hooks from the wall of the vessel.

According to still further features in the described preferred embodiments the medical device further comprises a thrust ring for thrusting the pressing ring.

According to still further features in the described preferred embodiments the medical device further comprises a lever for establishing contact between the thrust ring and the pressing ring.

According to still further features in the described preferred embodiments the medical device further comprises at least one adhesive layer for adhering at least two of: the non-woven liner, the non-woven cover and the at least one anastomotic member.

According to still further features in the described preferred embodiments the at least one adhesive layer is formed of a thermoplastic polymer having a characteristic melting temperature which is lower than characteristic melting temperatures of the electrospun fibers of each of the non-woven liner and the non-woven cover.

According to still further features in the described preferred embodiments the at least one adhesive layer comprises silicon.

According to still further features in the described preferred embodiments the electrospun fibers are made of a biocompatible polymer. According to still further features in the described preferred embodiments the electrospun fibers are manufactured from a liquefied polymer. According to still further features in the described preferred embodiments the electrospun fibers of the non-woven liner comprise polyethylene fibers and polyester fibers. According to still further features in the described preferred embodiments the electrospun fibers of the non-woven cover comprise polyethylene fibers and polyester fibers.

According to yet another aspect of the present invention there is provided a method of manufacturing a medical device for implantation in a vessel, the method comprising: providing at least one anastomotic member designed for engaging a wall of the vessel; electrospinning a first liquefied polymer on a precipitation electrode, thereby providing a non-woven liner of electrospun fibers; mounting the at least one anastomotic member onto the precipitation electrode; and electrospinning a second liquefied polymer on at least one of: the precipitation electrode, the non-woven liner and the at least one anastomotic member, so as to provide a non-woven cover of electrospun fibers.

According to further features in preferred embodiments of the invention described below, the electrospinning of the second liquefied polymer is done such that the at least one anastomotic member protrudes out of the non-woven cover.

According to still further features in the described preferred embodiments the electrospinning of the second liquefied polymer is done such that the at least one anastomotic member is flush with the non-woven cover.

According to still further features in the described preferred embodiments the method further comprises mounting at least one perforated member onto the non-woven cover, wherein the at least one perforated member is designed and constructed to receive protruding portions of the plurality of hooks.

According to still further features in the described preferred embodiments the method further comprises mounting a pressing ring on the non-woven cover, wherein the pressing ring is designed and constructed for bending protruding portions of the plurality of hooks. According to still further features in the described preferred embodiments the device further comprises mounting a thrust ring onto the non-woven cover, wherein the thrust ring is designed and constructed for thrusting the pressing ring. According to still further features in the described preferred embodiments the device further comprises connecting a lever to the thrust ring and the pressing ring.

According to still further features in the described preferred embodiments the method further comprises repeating the electrospinning of the first and the second liquefied polymers for different orientations of the precipitation electrode, so as to form a furcating structure having a plurality of tubular branches.

According to still further features in the described preferred embodiments the method further comprises applying pressure on at least one of the non-woven liner, the non-woven cover and the at least one anastomotic member.

According to still further features in the described preferred embodiments the method further comprises electrospinning a third liquefied polymer prior to the mounting of the anastomotic member, wherein a boiling point of the third liquefied polymer is higher than a boiling point of the first liquefied polymer.

According to still further features in the described preferred embodiments the method further comprises electrospinning a fourth liquefied polymer prior to the electrospinning of the second liquefied polymer, wherein a boiling point of the fourth liquefied polymer is higher than a boiling point of the second liquefied polymer.

According to still further features in the described preferred embodiments the method further comprises applying at least one adhesive layer on at least one of the non-woven liner and the at least one anastomotic member.

According to still further features in the described preferred embodiments the application of the at least one adhesive layer is effected by electrospinning.

According to still further features in the described preferred embodiments the at least one adhesive layer is formed of a thermoplastic polymer having a characteristic melting temperature which is lower than characteristic melting temperatures of the first and the second liquefied polymers.

According to still further features in the described preferred embodiments the method further comprises heating the device to a temperature which is above the characteristic melting temperature of the thermoplastic polymer and below the characteristic melting temperatures of the first and the second liquefied polymers.

According to still further features in the described preferred embodiments the applying the at least one adhesive layer comprises dipping the at least one anastomotic member in an adhesive solution prior to the mounting of the at least one anastomotic member. According to still further features in the described preferred embodiments the method further comprises heating the adhesive solution. According to still further features in the described preferred embodiments the adhesive solution comprises silicon.

According to still further features in the described preferred embodiments the method further comprises electrospinning an additional liquefied polymer on at least one of: the precipitation electrode, the non-woven liner, the at least one anastomotic member and the non-woven liner.

According to still further features in the described preferred embodiments at least one of the first liquefied polymer and the second liquefied polymer comprises a polyurethane.

According to still further features in the described preferred embodiments the additional liquefied polymer comprises a polyester.

According to still further features in the described preferred embodiments the additional liquefied polymer comprises a mixture of a polyester and a polyurethane.

According to still further features in the described preferred embodiments the electrospinning of the additional liquefied polymer and the electrospinning of the first liquefied polymer is substantially contemporaneously.

According to still further features in the described preferred embodiments the electrospinning of the additional liquefied polymer and the electrospinning of the second liquefied polymer is substantially contemporaneously.

According to still further features in the described preferred embodiments the electrospinning of the additional liquefied polymer and the electrospinning of the first liquefied polymer is performed alternatingly.

According to still further features in the described preferred embodiments the electrospinning of the additional liquefied polymer and the electrospinning of the second liquefied polymer is performed alternatingly.

According to still further features in the described preferred embodiments the electrospun fibers of the non-woven liner and/or the non-woven cover are aligned at a predetermined orientation relative to a longitudinal axis of the device.

According to still further features in the described preferred embodiments the electrospun fibers of the non-woven liner and/or the non-woven cover are randomly aligned.

According to still further features in the described preferred embodiments at least a portion of the electrospun fibers of the non-woven liner and/or the non-woven cover are aligned substantially along a circumferential direction of the non-woven liner and/or the non-woven cover.

According to still further features in the described preferred embodiments at least one of the non-woven liner and the non-woven cover comprises at least one medicament incorporated therein, for delivery of the at least one medicament into a body vasculature during or after implantation of the device within the body vasculature.

According to still further features in the described preferred embodiments the expandable supporting element is designed and constructed for dilating a constricted blood vessel in a body vasculature.

According to still further features in the described preferred embodiments the non-woven liner and the non-woven cover are each independently characterized by a porosity of at least 50%.

According to still further features in the described preferred embodiments the non-woven liner and the non-woven cover are each independently characterized by a porosity of from about 50% to about 85%.

According to still further features in the described preferred embodiments a thickness of the electrospun fibers is from about 100 nanometers to about 500 nanometers.

According to still further features in the described preferred embodiments at least a portion of the electrospun fibers is made of a biodegradable polymer.

According to still further features in the described preferred embodiments at least a portion of the electrospun fibers is made of a biostable polymer.

According to still further features in the described preferred embodiments at least a portion of the electrospun fibers is made of a combination of a biodegradable polymer and a biostable polymer.

According to still another aspect of the present invention there is provided an anastomosis procedure, comprising: providing a medical device for implantation in a vessel as described herein; forming a plurality of vessel openings; and connecting the medical device to the plurality of vessel openings such that each vessel opening is engaged by one end of the at least one anastomotic member.

According to further features in preferred embodiments of the invention described below, the anastomosis procedure further comprises expanding the expandable supporting elements such that a pressure is generated between the non-woven cover and the plurality of vessel openings.

According to still further features in the described preferred embodiments the anastomosis procedure further comprises piercing the wall of at least one vessel using the plurality of hooks, thereby mounting the device to the at least one vessel.

According to still further features in the described preferred embodiments the piercing comprises piercing an inner wall of at least one vessel such that plurality of hooks protrudes out of an outer wall of the at least one vessel.

According to still further features in the described preferred embodiments the anastomosis procedure further comprises receiving protruding portions of the plurality of hooks using at least one perforated member mounted on the non-woven cover.

According to still further features in the described preferred embodiments the anastomosis procedure further comprises bending protruding portions of the plurality of hooks using a pressing ring.

According to still further features in the described preferred embodiments the anastomosis procedure further comprises thrusting the pressing ring using a thrust ring. According to still further features in the described preferred embodiments the thrusting is by a lever.

According to still further features in the described preferred embodiments at least one opening of the plurality of vessel openings is formed on a side of a vessel.

According to still further features in the described preferred embodiments at least one opening of the plurality of vessel openings forms a vessel end.

According to still further features in the described preferred embodiments the anastomosis procedure further comprises introducing at least one balloon into a lumen of at least one vessel, and inflating the balloon so as to reduce flow of fluids into regions being nearby to at least one opening of the plurality of vessel openings.

According to still further features in the described preferred embodiments the anastomosis procedure further comprises applying an under pressure substantially contemporaneously with the formation of the plurality of vessel openings, so as to remove fluids and tissue debris from regions being nearby to at least one opening of the plurality of vessel openings.

The present invention successfully addresses the shortcomings of the presently known configurations by providing devices, kit and methods suitable for performing anastomosis procedure. The devices, kit and methods of the present invention enjoy properties far exceeding the prior art.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1a-b are schematic illustrations of a longitudinal cross-section (FIG. 1a) and a transversal cross-section (FIG. 1b) of a medical device for implantation in a vessel, according to a preferred embodiment of the present invention;

FIG. 2a is a schematic illustration of a side view of a medical device for implantation in a vessel in which an anastomotic member protrudes from its liner and cover, according to a preferred embodiment of the present invention;

FIG. 2b is a schematic illustration of the medical device of FIG. 2a, after end-to-end anastomosis procedure, according to a preferred embodiment of the present invention;

FIG. 3 is a schematic illustration of a medical device for implantation in a vessel in a preferred embodiment in which the device forms a furcating structure;

FIGS. 4a-b are schematic illustrations of a longitudinal cross-section (FIG. 4a) and a transversal cross-section (FIG. 4b) of a medical device engaging a wall of a vessel from the side, in a preferred embodiment in which a star-shape structure is employed to anchor the medical device to the wall;

FIGS. 5a-f are schematic illustrations explaining the procedure of side-to-end implanting of a medical device a vessel, in preferred embodiments in which the device serves for end-to-side anastomosis;

FIG. 6 is a schematic illustration of a “T”-shape medical device for implantation in a vessel, in which one end can be used for end-to-side anastomosis and two other ends can be used in end-to-end anastomosis, according to a preferred embodiment of the present invention;

FIG. 7 is a is a schematic illustration of an accessory device for forming an opening in a vessel, according to a preferred embodiment of the present invention;

FIGS. 8a-b are schematic illustrations showing a technique for reducing flooding of an operation area, according to a preferred embodiment of the present invention; and

FIG. 9 is a flowchart diagram of a method of manufacturing a medical device for implantation in a vessel, according to a preferred embodiment of the present invention.

### DESCRIPTION OF THE PREFERRED EMBODIMENTS

The present invention is of a device and method which can be used in vessel implantation procedures. Specifically, the present invention can be used to replace, bypass or connect blood vessels and other fluid-transporting vessels of the body, for example, coronary arteries, peripheral blood vessels, urinary vessels and the like.

The principles and operation of a device and methods according to the present invention may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

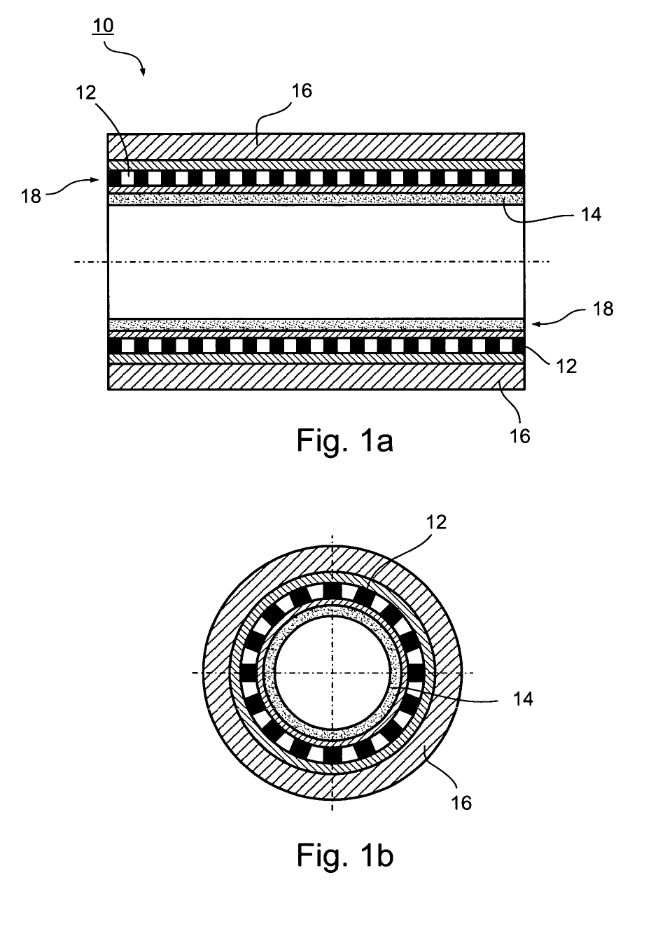

Referring now to the drawings, FIGS. 1a-b illustrate a longitudinal cross-section (FIG. 1a) and a transversal cross-section (FIG. 1b) of a medical device 10 for implantation in a vessel, according to a preferred embodiment of the present invention. Device 10 is preferably tubular and comprises one or more anastomotic members 12, a non-woven liner 14 of electrospun fibers and a non-woven cover 16 of electrospun fibers. Anastomotic member(s) 12 preferably interpose liner 14 and cover 16.

The electrospun fibers forming liner 14 and/or cover 16 are preferably made of a biocompatible polymer, such as, but not limited to, a biodegradable polymer, a biostable polymer or a combination thereof. For example, according to various exemplary embodiments of the present invention electrospun fibers are made of a polyurethane (e.g., polyether urethane, polycarbonate urethane, siloxane-based aromatic polycarbonate urethane) found in, e.g., International Patent Application Nos. WO 91/00270, WO 99/00236 and WO 98/00497). More preferably, liner 14 and/or cover 16 also comprise electrospun fibers made of a polyester (e.g., polyethylene terephthalate). The advantage of using a combination of fibers made of a polyurethane with fibers made of a polyester, is that polyurethane fibers provide the liner and cover with sealing and non-thrombogenic properties, while polyester fibers provide the liner and cover with durable high tensile strength.

According to a preferred embodiment of the present invention liner 14 and/or cover 16 comprise one or more medicament incorporated therein, for a delivery of the medicament(s) into the vasculature during or after implantation. It is appreciated that the drug incorporated, as well as the concentration and method of incorporation into the device is in accordance with the type of vessel being replaced, and with the particular pathology of the patient. The preferred mechanism of medicament release from liner 14 and/or cover 16 is by diffusion, regardless of the technique employed to embed the medicament therein. The duration of medicament release in a predetermined concentration depends on several variants, which may be controlled during the manufacturing process. One variant is the chemical nature of the carrier fiber and the chemical means binding the medicament to it. This variant may be controlled by a suitable choice of the polymer(s) used in the manufacturing process. Another variant is the area of contact between the body and the medicament, which can be controlled by varying the free surface of the electrospun fibers. Also affecting the duration of medicament release is the method used to incorporate the medicament within liner 14 and/or cover 16, as is further detailed hereinunder.

The porosity, thickness and materials of liner 14 and cover 16 are preferably selected so as to optimize the biocompatibility of device 10. For example, the porosity, thickness and materials of liner 14 is preferably selected to prevent bleeding, preclude preclotting and/or allow efficient endothelization. Additionally, according to various exemplary embodiments of the present invention, thickness and materials of cover 16 are selected so as to provides requisite mechanical properties, specifically high compliance and high breaking strength.

Generally, liner 14 and/or cover 16 are characterized by a porosity of at least 50%, more preferably from about 50% to about 85%, a thickness of from about 50 μm to about 1000 μm, average fibers thickness of from about 100 nanometers to about 500 nanometers. Preferably, but not obligatory, the porosity and thickness of cover 16 is larger than the porosity and thickness of liner 14.

An additional characteristic which affect the mechanical properties of device 10 is the orientation of the electrospun fibers. As further detailed hereinunder the manufacturing process of device 10 can be adapted in accordance with the desired orientation of the electrospun fibers. Preferred orientations along which the electrospun fibers are aligned include, without limitation, random alignment and circumferential alignment.

Optionally and preferably, device 10 further comprises one or more adhesive layers 15, for adhering liner 14 to cover 16, liner 14 to member 12 and/or member 12 to cover 16. Adhesive layer(s) 15 can be made of any adhesive material known in the art, preferably a biocompatible adhesive material, such as, but not limited to, a thermoplastic polymer and silicon. A method of applying layer(s) 15 is provided hereinafter.

Member 12 can be a metal wire, a metal cylinder having diversified shaped orifices therein or any polymer agent having sufficient mechanical strength to mount device 10 on the vessel. Suitable members include, without limitation, members known as expandable supporting elements or stents which are found, e.g., in U.S. Pat. Nos. 6,019,789, 4,655,771, 5,092,877, 5,226,913, 5,741,353 and 5,849,057 the contents of which are hereby incorporated by reference. In the embodiments in which anastomotic member is a stent, member 12 can dilate a constricted blood vessel in the body vasculature. In this embodiment, member 12 is radially expandable. The expansibility of member 12 may be achieved by providing member 12 in a form of a deformable mesh of metal wires, which can be, for example, a deformable mesh of stainless steel wires. Hence, when device 10 is placed in the desired location in an artery, member 12 expands radially to substantially dilate the arterial tissues surrounding device 10 to eradicate a flow constriction in the artery. The expansion may be performed by any method known in the art, for example by using a balloon catheter or by forming member 12 from a material exhibiting temperature-activated shape memory properties, such as Nitinol. As stated, member 12 at least partially interposes liner 12 and cover 14. Therefore, liner 12 and/or cover 14 are preferably fabricated from non-woven elastomeric fibers, more preferably elastomeric polymer fibers, which stretch as member 12 radially expands.

According to a preferred embodiment of the present invention both liner and cover are preferably sufficiently elastic to sustain, without breakage and/or excessive resistance, the expansion of the anastomotic member which is typically from about 200% to about 400% in diameter. In addition, the cover preferably has high tensile strength to withstand high fluid pressure in the vessel. For example, when the device is implanted in the aorta, the cover has a tensile strength which is sufficient to withstand the aortic blood pressure without the formation of a recurrent aneurysm.

As stated, the use of polyurethane fibers in combination with polyester fibers provides sealing and non-thrombogenic properties on the one hand, and durable high tensile strength on the other hand. Such combination is preferably realized by a polyurethane matrix in which polyester fibers are distributed. The polyester fibers can be interconnected into a spatial mesh of the polyurethane fibers, or be positioned isolated in the matrix. Additionally, the polyester fibers can be either distributed uniformly within the matrix volume, or form a sparse bi-dimensional structure.

Upon expansion of the anastomotic member, the matrix experiences radial tensile forces, and, while the anastomotic member is shortened, axial compressing loads also take place. The polyester fibers and the polyurethane fibers tend to align along the force vector, resulting in a partially change of the fiber orientation. The extent of orientation on exposure to tensile loads depends on the number and density of contact points between the fibers. Due to the friction forces in the contact points, the re-orientation of the fibers is accompanied by stretching of the fibers, hence also of the entire matrix. The elongation of the fibers and matrix is directly proportional to the loading, and it prolongs until they reaches their tensile strength limit. Being less prone to tension, the polyester fibers restrain further deformation. Thus, large percentage of polyester fibers, corresponds to a lesser extent of matrix elongation and a higher tensile strength.

According to a preferred embodiment of the present invention the percentages of polyester fibers and polyurethane fibers are selected so as to optimize both elasticity (provided by the polyurethane fibers) and rigidity (polyester fibers) of the liner and cover. This can be achieved by selecting the percentages such that the elastic properties of the liner and cover match the elastic properties of the anastomotic member. In other words, the amount of polyester fibers is selected such that when the anastomotic member completes its full expansion, the polyester fiber are stretch to their maximal or close to maximal extent.

The optimal volumetric amount of polyester fiber also depends on the number of contact points among the fibers which is characterized by the porosity of the liner and cover. When the number of contact points is small, the friction forces between fibers are also small and the fibers can more freely change their orientation. Conversely, when the number of contact points is large, the re-orientation of the fibers is constrained by the friction forces, and the elongation is more pronounced. Therefore, according to a preferred embodiment of the present invention when the porosity of the liner and cover is high, larger amount of polyester fibers can be used to achieve the required rigidity. In experiments performed by the Inventors of the present invention it was found that a preferred porosity of the liner and cover is about 60-70%, and a preferred percentage of polyester fibers is about 15-30% by volume, where the high values (70% porosity and 30% polyester) corresponds to the axial orientation of polyester fibers, and the low values (60% porosity and 15% polyester) correspond to circumferential orientation thereof.

Referring now again to the drawings, in the embodiment shown in FIG. 1a anastomotic member 12 fully superimposes liner 14 and cover 16. However, this need not necessarily be the case, since, for some applications, it may not be necessary for anastomotic member to extend along the length of liner 14 and cover 16.

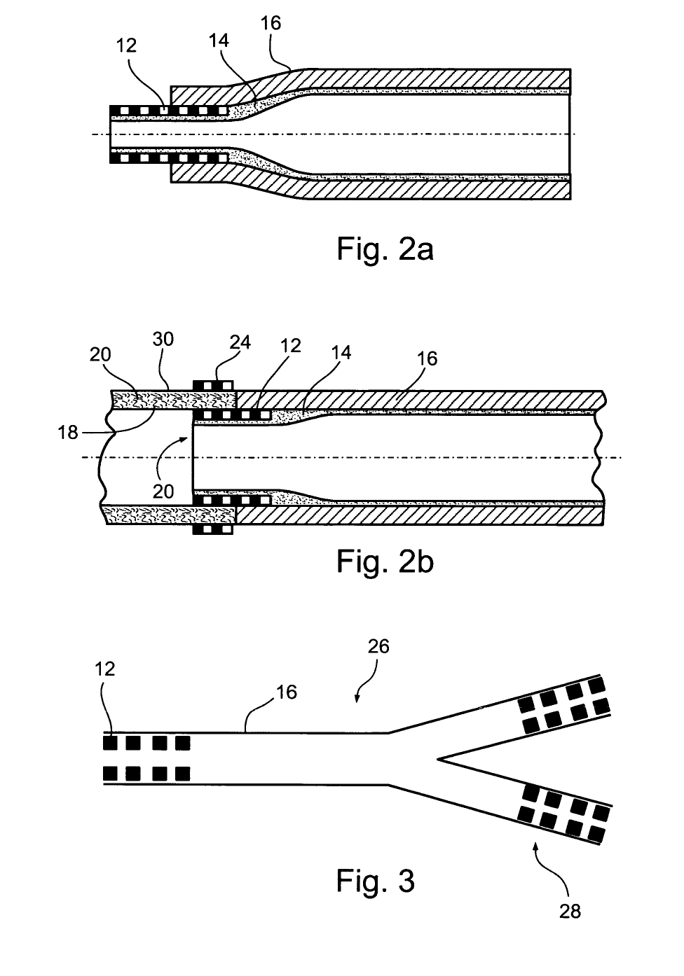

An alternative, yet preferred, embodiment is shown in FIGS. 2a-b. In this embodiment, member 12 partially interposes liner 14 and cover 16.

With reference to FIG. 2a, anastomotic member 12 preferably protrudes out of cover 14 and/or liner 12. Such protrusion facilitates the mounting of device 10 to the vessel, preferably without or with minimal sewing. Thus, according to various exemplary embodiments of the present invention, anastomotic member 12 is designed for engaging one or more ends of device 10 to a wall of the vessel upon implantation of device 10 within the vessel.

FIG. 2b is a schematic illustration of device 10 in a preferred embodiment in which end 22 of device 10 engages an inner wall 18 of a vessel 20. As shown in FIG. 2b, anastomotic member 12 is in contact with inner wall 18 thus fixing device 10 in its proper place, while cover 16 is flush with an outer wall 30 of vessel 20. Thus, the present embodiment successfully enables the attachment of device 10 with vessel 20 while ensuring a smooth joining between device 10 and vessel 20. Preferably, a pressing ring 24 is mounted on outer wall 30 such that the wall of vessel 20 is subjected to pressure both from its inner side 18 (via forces applied by member 12) and its outer side 30 (via forces applied by ring 24). Ring 24 can be made of an elastic biocompatible material (e.g., an elastic polymer), a metal, a mesh of wires or any other structure which is capable of applying an inward, radial-oriented, force.

Many shapes of device 10 are contemplated, depending on the medical procedure in which device 10 is used. Preferably, the shape of device 10 is selected in accordance with the type of vessel (e.g., aorta, carotid, coronary artery, etc.) and the type of anastomosis required (e.g., end-to-side anastomosis, end-to-end anastomosis).

Reference is now made to FIG. 3 which is a schematic illustration of a portion device 10 in a preferred embodiment in which device 10 forms a furcating structure 26 having a plurality of tubular branches 28. In the exemplifying embodiment shown in FIG. 3a, device 10 forms a “Y”-shape, i.e., a bifurcating structure with three ends. But this need not necessarily be the case since, for some applications, it may be desired to have other shapes of bifurcating structure (e.g., a “T”-shape) or other furcating structures (e.g., trifurcating structures). Additionally, each branch of device 10 can be shaped differently so as to allow the use of different branches for different types of anastomoses or different vessels.

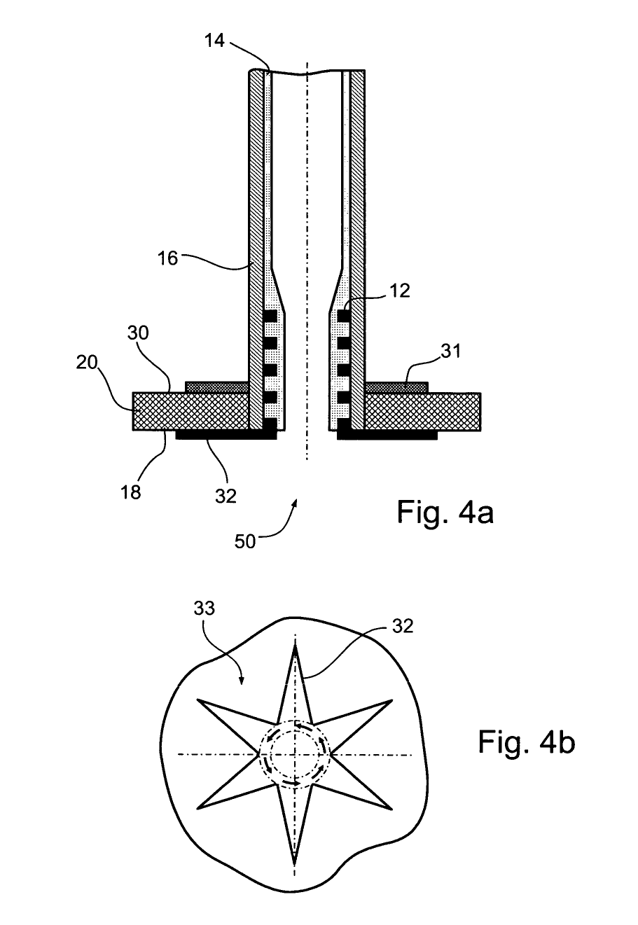

Reference is now made to FIGS. 4a-b which are schematic illustrations of device 10 in a preferred embodiment in which device 10 serves for end-to-side anastomosis. These embodiments can be used, for example, when device 10 is used during a heart bypass procedure in which case device 10 channels the blood flow around the blocked portions of the coronary arteries. Thus, one end of device 10 can be connected to the side of the aorta and the other end(s) can be connected to the side or end of one or more diseased coronary arteries. According to the presently preferred embodiment of the invention member 12 is expandable and comprises a plurality of hooks 32, which may form, for example, a star-shape structure 33. In use, once end 50 of device 10 is positioned in an opening in the wall of vessel 20, member 12 and star-shape structure 33 preferably expand, such that member 12 is pressed against the wall of vessel 20 and star-shape structure 33 anchors device 10 in its position. In various exemplary embodiments of the present invention, an additional circular member 31 (which may be, for example, similar to pressing ring 24) is mounted on cover 16 so as to prevent device 10 from moving inward into the lumen of vessel 20.

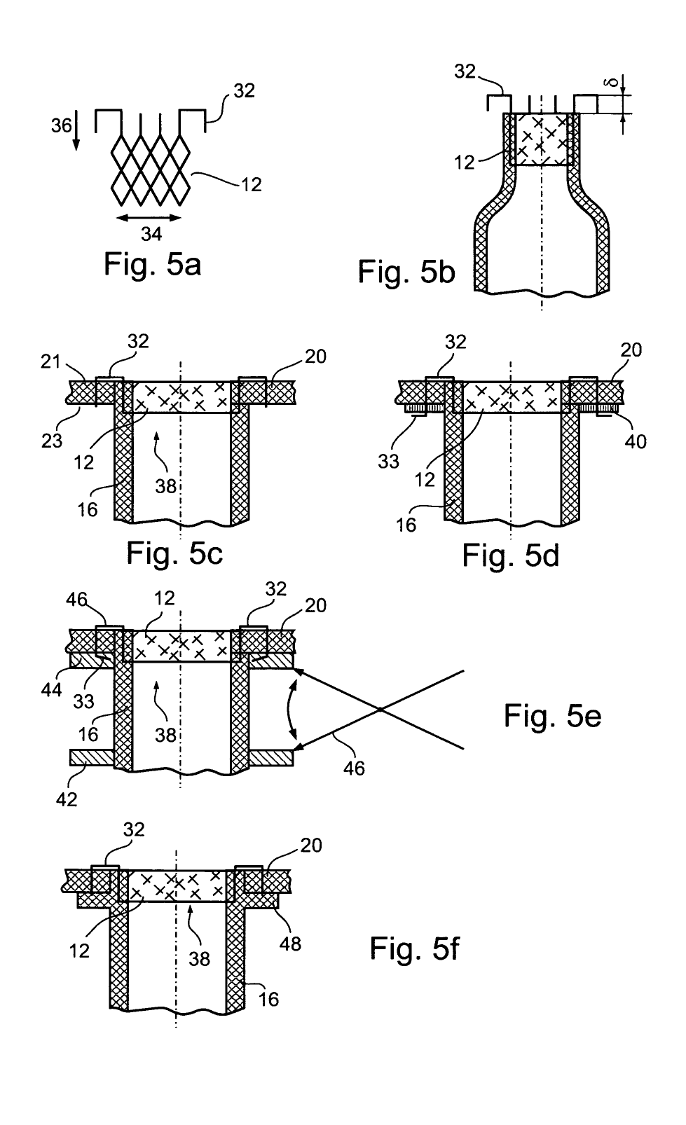

Reference is now made to FIGS. 5a-f which are schematic illustrations explaining the procedure of side-to-end implantation of device 10, according to various exemplary embodiments of the present invention.

Referring to FIG. 5a, according to various exemplary embodiments of the present invention member 12 is manufactured as a rhomboidal mesh of wires (e.g., a stent), capable of expanding radially and equipped with hooks 32. The number of hooks preferably depends on the diameter of member 12. Typically, from 2 to 10 hooks, more preferably from 6 to 12 hooks are used. Hooked 32 is preferably fixed to the upper nodular parts of the rhomboids such that when member 12 expands to a direction designated by double headed arrow 34, hooks 32 move to the direction designated by arrow 36.

FIG. 5b illustrates device 10 in a pre-implantation state, i.e., prior to its connection to the vessel wall. In this state, member 12 is still in its reduced diameter and the level of hooks 32 slightly exceeds cover 16. The height of hooks 32 above cover 16 (designated 8 in FIG. 5b) is preferably from about 1 mm to about 6 mm.

As used herein the term “about” refers to ±10%.

FIG. 5c illustrates the implantation of device 10 within vessel 20. An opening 38 is made in the wall of vessel 20 such that its diameter insignificantly exceeds outer diameter of device 10 in its pre-implantation state. Device 10 is introduced into opening 38 and member 12 expands (e.g., using a catheter balloon). As member 12 expands, cover 16 come into tense contact with the wall of opening 38 thus sealing it. At the same time, hooks 32 move towards vessel 20 pierce its inner wall 21 and protrude out of its outer wall 23. The displacement of hooks 32 can be further enhanced by pulling device 10 outside opening 38 once member 12 is in its expanded state.

As will be appreciated by one of ordinary skill in the art, in its final position, device 10 cannot come out of the opening, as it is held by hooks 32. Additionally, device 10 is prevented from falling into the vessel by the pressure fluids (e.g., blood) flowing through the vessel, and by a friction force between the wall of the vessel and cover 16 which is enhanced by the pressure applied by member 12. The connection reliability can be further increased by bending the protrusion of hooks 32 or by employing more hooks or other elements, e.g., discs, rings and the like.

FIG. 5d illustrates one preferred embodiment in which a perforated member 40 (e.g., a disc) is utilized for fixing device 10 to vessel 20. Hence, according to the presently preferred embodiment of the invention, protrusions 33 of hooks 32 can be passed through holes in member 40, so as to prevent detachment of hooks 32 from vessel 20. Member 40 can be made, for example, of metal or polymer mesh.

FIG. 5e illustrate an additional embodiment in which a thrust ring 42 and/or a pressing ring 44 are utilized for fixing device 10 to vessel 20. Thrust ring 42 is preferably fixed to cover 16 (e.g., using a balloon), and pressing ring 44 is positioned closed to the protruding portions 33 of hooks 32. Pressing ring 44 is preferably equipped with a groove 46 shaped so as to grasp and bend protruding portions 33, as a predetermined angle (say, bending of about 40-70 degrees). Preferably, a triangle-sectioned groove is employed. A contact between pressing ring 44 and thrust ring 42 can then be established, for example, using a pair of levers 46 which move 44 in the direction of 42.

FIG. 5f illustrates another preferred embodiment in which device 10 comprises a ring-shaped protuberance 48. Protuberance 48 can be integrated with or attached to cover 16. Upon expansion of member 12, hooks 32 pierce the wall of vessel 20, and protuberance 48 prevents, at least partially, the displacement of device 10 inside opening 38.

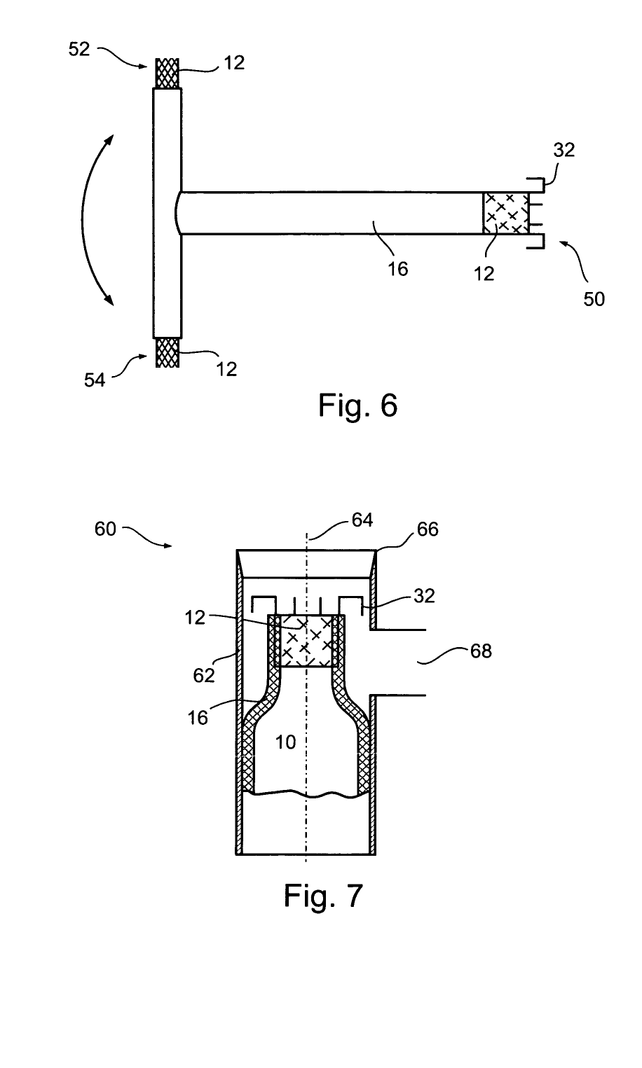

As stated, device 10 can form a furcating structure, whereby different branches of device 10 can be used for different anastomoses or different vessels. One such embodiment is shown in FIG. 6.

FIG. 6 schematically illustrate a preferred embodiment of the present invention in which device 10 forms a “T”-shape, having a first end 50 a second end 52 and a third end 54. In the exemplified embodiment shown in FIG. 6, end 50 serves for end-to-side anastomosis and ends 52 and 54 serve for end-to-end connection. This embodiment is particularly useful for heart bypass procedure, whereby end 50 is connected to the aortal wall, e.g., using hooks 32 as further detailed hereinabove, and ends 52 and 54 are connected to ends of the diseased coronary arteries.

One of the difficulties for a surgeon performing a bypass operation, or any other operation in which the aorta is opened while the heart is beating, is the high aortic blood pressure. Upon the formation of the opening of the aortal wall, blood starts to flow through the opening resulting in sever blood loss. The present embodiments successfully provide an accessory device 60 for forming an opening in a vessel, while substantially reducing or preventing the flooding of the operation area by blood.

Reference is now made to FIG. 7 which is a schematic illustration of accessory device 60, according to a preferred embodiment of the present invention. One or more units of device 60 can be presented, if desired, together with one or more units of device 10, in a pack or dispenser device, such as an FDA approved kit. The pack may be accompanied by instructions for use. The pack may also be accommodated by a notice in a form prescribed by a governmental agency.

Hence, device 60 preferably comprises a tubular encapsulation 62 for encapsulating device 10, preferably in a manner such that device 10 is movable along a longitudinal axis 64 of encapsulation 62. Encapsulation 62 is preferably made of metal (e.g., stainless steel) or any other material (e.g., a polymer) having a sufficient strength to withstand aortic blood pressure. According to various exemplary embodiments of the present invention device 60 further comprises a generally circular cutting member 66 integrated with or attached to encapsulation 62. Additionally device 60 comprises a vacuum channel 68, preferably formed on the side of tubular encapsulation 62.

In use, member 12 is preferably expanded (e.g., using a balloon), until cover 16 is pressed to the inner wall of encapsulation 62 to form a hermetically sealed connection therebetween. Subsequently, cutting member 66 is brought into contact with the vessel wall (not shown) to form an opening therein. Cutting member 66 can form the opening in the vessel wall using any technique known in the art, such as, but not limited to, cutting and burning. At the same time, or shortly thereafter, an under pressure is formed in vacuum channel 68, e.g., using a pump (not shown), so as to channel an efflux of biological material (tissue debris, blood) from encapsulation 62, thereby to clear the operation area.

Once the opening is formed, the diameter of member 12, hence also of cover 16, can be reduced (e.g., by deflating the balloon), and device 10 can be advanced within encapsulation 62 so as to engage the opening.

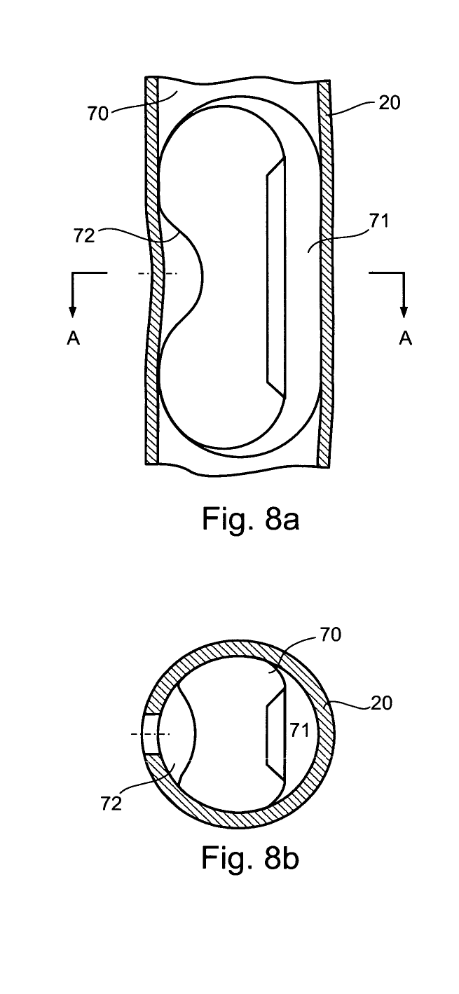

FIGS. 8a-b illustrate an alternative embodiment for reducing flooding of the operation area, where FIG. 8b is a cross-sectional view along the cut designated A-A in FIG. 8a. In this embodiment, a balloon 70 is inserted to the lumen of the vessel. For example, when the vessel is a blood vessel, balloon 70 can be inserted from a far location using a catheter inserted, e.g., through an incision in the skin of the groin and the wall of the femoral artery. Balloon 70 is preferably shaped so as to prevent fluid communication between operation area 72 and an unblocked portion 71 of the lumen. More specifically, upon the inflation of balloon 70, fluids (e.g., blood) continue to flow through portion 71 of the lumen, substantially without flooding operation area 72, which is at least partially isolated therefrom.

According to another aspect of the present invention there is provided a method of manufacturing a medical device for implantation in a vessel. The method can be used, for example, for manufacturing medical device 10.

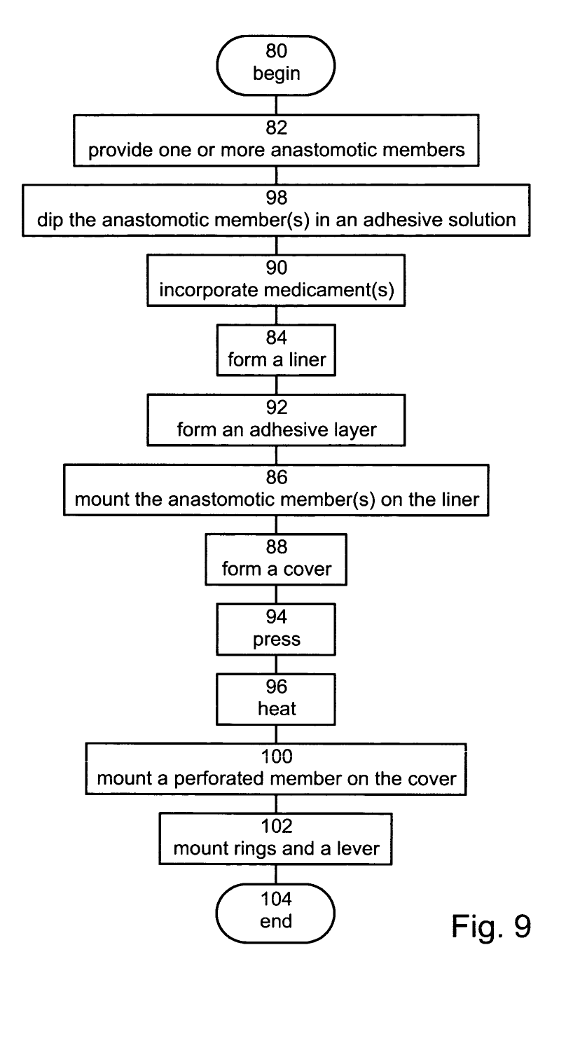

Reference is now made to FIG. 9 which is a flowchart diagram of the method, according to various exemplary embodiments of the present invention. It is to be understood that, unless otherwise defined, the method steps described hereinbelow can be executed either contemporaneously or sequentially in many combinations or orders of execution. Specifically, the ordering of the flowchart of FIG. 9 is not to be considered as limiting. For example, two or more method steps, appearing in the following description or in the flowchart of FIG. 9 in a particular order, can be executed in a different order (e.g., a reverse order) or substantially contemporaneously. Additionally, selected method steps, appearing, for the sake of brevity only once in FIG. 9, can be can be executed a plurality of times (e.g., twice).

Referring to FIG. 9, the method begins at step 80 and preferably proceeds to step 82 in which one or more anastomotic members are provided. Preferably, one or more units of anastomotic member 12 are provided. The method continues to step 84 which represents an electrospinning process in which a first liquefied polymer is dispensed on a precipitation electrode so as to provide a non-woven liner, such as liner 14. The diameter of the precipitation electrode is preferably from about 10 mm to about 30 mm.

According to a preferred embodiment of the present invention the precipitation electrode is shaped in accordance with the desired shape of the device. For example, for a tubular device (see, e.g., FIGS. 1a-2b) a tubular precipitation electrode is used; for a furcating device, a furcating (e.g., bifurcating, trifurcating) precipitation electrode is used. In any event, the electrospinning process is executed such that all sides of the precipitation electrode are covered by electrospun fiber. As will be appreciated by one ordinarily skilled in the art, this can be done by rotation the precipitation electrode or the mechanism from which the liquefied is dispensed. The rotation can be done continuously while the liquefied polymer is dispensed, or in a discrete manner such that the electrospinning is repeated a plurality of times, each time for different orientation of the precipitation electrode.

When the precipitation electrode is tubular, it is sufficient to rotate the electrode about its longitudinal axis. When a furcating precipitation electrode is used, several axes of rotation are preferably employed. For example, a longitudinal axis and an axis perpendicular to the longitudinal axis can be used. The method preferably proceeds to step 86 in which the anastomotic member(s) are mounted onto the precipitation electrode, such that at least a portion of each anastomotic member superimposes at least a portion of the liner, as further detailed hereinabove (see, e.g., FIG. 1a for the embodiment in which the anastomotic member is flush with the liner and FIG. 2a for the embodiment in which the anastomotic member protrudes out of the liner).

The method preferably continues to step 88 which represents an additional electrospinning process in which a second liquefied polymer is dispensed so as to form non-woven cover of electrospun fibers. The cover preferably has the characteristics of cover 16 as further detailed hereinabove. The second liquefied polymer can be dispensed on the precipitation electrode, the liner and/or the anastomotic member(s), depending on the level of superimposing between the cover, the liner and the and anastomotic member(s). Optionally and preferably, step 88 is accompanied by a formation of a ring-shaped protuberance (e.g., protuberance 48, see FIG. 5f) onto the cover. This can be done by increasing the amount of fibers precipitation in a predetermined location, so as to thicken the cover thereby to form the protuberance. The method preferably comprises a step 94 in which a pressure is applied so as to achieve higher level of adherence between the liner, cover and anastomotic member. Step 94 can be executed more than once. Preferably, but not obligatory, step 92 is executed a first time subsequently to step 84 and a second time subsequently to step 86.

The electrospinning steps may be performed using any electrospinning apparatus known in the art. According to the electrospinning method, the liquefied polymer is charged and drawn into a dispensing electrode, and then, subjected to an electric field, dispensed in a direction of the precipitation electrode. Moving with high velocity in the inter-electrode space, jets of liquefied polymer evaporate, thus forming fibers which are collected on the surface of the precipitation electrode. A typical thickness of the fibers thus formed ranges between 100 nanometers to about 500 nanometers.

It was found by the Inventor of the present invention that randomized structure surface comprising 100-200 nm fibers possesses non-thrombogenic properties. Devices with such inner surface are capable of efficient endothelization and have good general coalescence. Additionally it was found by the Inventor of the present invention that a liner and/or the cover thickness of less than 400-500 μm with about 90% of the fibers having thickness of about 1-2 μm, have good sealing properties and prevents blood leakage.

The orientation of the fibers can be controlled by varying the direction of the electric field (e.g., using more than one electrode). As stated, such predetermined orientation affects the mechanical characteristics of the device.

The advantage of using the electrospinning method for fabricating the medical device is flexibility of choosing the polymer types and fibers thickness, thereby providing a final product having the required combination of strength, elastic and other properties as delineated herein. In addition, the electrospinning method has the advantage of allowing the incorporation of various chemical components, such as medicaments, to be incorporated in the fibers, e.g., by dissolving or dispersing such medicaments in the liquefied polymers prior to electrospinning.

Thus, according to a preferred embodiment of the present invention, the method may further comprises an optional step 90 in which one or more medicaments are incorporated within the liquefied polymers. Step 90 can be executed more than once. Preferably, but not obligatory, step 90 is executed a first time in combination (e.g., prior, subsequently or contemporaneously) with step 84 and a second time in combination with step 86. The medicament(s) may be of any type which can be useful in treating the lumen of the vessel. For example, when the vessel is a blood vessel, anti-platelets and anticoagulants can be incorporated to control platelet aggregation and adhesion; and growth factor, receptor blockers and antagonists may be used to limit the normal repair response. Preferably, but not obligatory, the medicament is loaded into biodegradable polymer fibers, which are preferably axially oriented hence do not essentially contribute to the radial strength. Such incorporation of medicaments results in slow release of drugs upon biodegradation of the fibers. The medicament can also be in a powder form or micro-encapsulated particulates form so that it can be sprayed as a shower of particles onto a liner 14 and/or cover 16, depending on the scheduling of step 90. Representative examples of medicaments which can be incorporated in the liner and/or cover include, without limitation, heparin, tridodecylmethylammonium-heparin, epothilone A, epothilone B, rotomycine, ticlopidine, dexamethasone, caumadin, and other pharmaceuticals falling generally into the categories of antithrombotic drugs, estrogens, corticosteroids, cytostatics, anti-coagulant drugs, vasodilators, and antiplatelet drugs, trombolytics, antimicrobials or antibiotics, antimitotics, antiproliferatives, antisecretory agents, nonsterodial antiflammentory drugs, grow factor antagonists, free radical scavengers, antioxidants, radiopaque agents, immunosuppressive agents and radio-labeled agents.

One or more of the electrospinning steps (steps 84 and 88) preferably comprises the use of two different polymers. For example, according to various exemplary embodiments of the present invention the liner and/or cover are made of polyurethane fibers as well as polyester fibers, so as to achieve the aforementioned properties of elasticity and rigidity. According to a preferred embodiment of the present invention the same or similar solvents are used for liquefying the polyurethane and polyester. The advantage of this embodiment is that the use of similar or identical solvents facilitates the bonding between the polyurethane fibers and the polyester fibers. Preferred solvents for liquefying the polymers include, without limitation, solvents which are based on phenol, methylene chloride and trifluoroacetic acid. While reducing the preset invention to practice it was uncovered that the bonding strength between the polyurethane matrix and polyester fiber is significantly increased when a small amount of polyurethane (e.g., from about 10% to about 20%) is added to polyester solution.

According to a preferred embodiment of the present invention the method comprises an additional step 92 in which one or more adhesive layers are formed on the liner, the anastomotic member and/or the liner. Step 92 can be executed more than once. Preferably, but not obligatory, step 92 is executed a first time subsequently to step 84 and a second time subsequently to step 86. The formation of adhesive layer can be effected from several procedures in any combination.

Hence, in one such procedure, the final stage of liner generation and the initial stage of cover generation are preferably conducted in an elevated productivity, typically from about 1.5-fold productivity to about 3-fold productivity. Elevated productivity can be achieved, for example, by providing higher pressure or higher amount of liquefied polymer in the electrospinning process. This procedure can also include the replacement of the liquefied polymers (the polymer types and/or the solution in which they are dissolved). It was found by the Inventor of the present invention that the use of liquefied polymer with higher boiling point in the final stage of liner generation provides good adhesive properties which are preserved during the mounting of the anastomotic member. Similarly, the use of liquefied polymer with higher boiling point in the final stage of liner generation provides good adhesive properties between the anastomotic member and the cover.

In another such procedure, an intermediate adhesion layer is preferably electrospun prior to- and/or immediately after the execution of step 86. The intermediate adhesion layer is preferably made of a thermoplastic polymer having a characteristic melting temperature which is considerably lower than the characteristic temperature of the first and second polymers used for the liner and cover. The present procedure is preferably supplemented by step 96 in which, once the electrospinning processes are completed, the device is heated to a temperature which is above the characteristic melting temperature of the intermediate adhesion layer but below the characteristic melting temperature of the liner and the cover. The heating results in a conversion of the intermediate adhesion layer to a viscous-flowing state and promotes the adhesion of the anastomotic member to the liner and cover.

For example, the liner and cover can be made of polycarbonate urethane with melting point of about 230° C., and the intermediate adhesion layer can be made of polycarbonate urethane with melting point of about 160° C. In this example, the device can be heating to a temperature of about 160-170° C., resulting in the desired adherence effect.

An additional procedure suitable for achieving adherence between the liner and cover and the anastomotic member, is represented by step 98, which is preferably executed prior to the mounting of the anastomotic member on the precipitation electrode. In step 98, the anastomotic member is preferably dipped into an adhesive solution, such as, but not limited to, a silicon rubber solution. The dipping is preferably supplemented by a thermal treatment of the anastomotic member so as to allow chemical cross-linking between the anastomotic member and the adhesive solution. For the case of silicon rubber solution, the heating can be to a temperature of 100-110° C. for about 30 minutes.

In a further step 100 of the method, one or more perforated members are mounted onto the cover. The perforated member(s) are preferably several units of perforated members 40 as further detailed hereinabove. The method can also comprise (in addition or as an alternative to step 100) a step 102 in which a pressing ring (e.g., pressing ring 44) and, optionally a thrust ring (e.g., thrust ring 42) and a lever (e.g., lever 46) can be mounted on the cover, preferably according to the construction shown in FIG. 5e and further detailed hereinabove.

Following is a non exhausted list of biostable polymers which can be used in the liner and/or cover. Polycarbonate based aliphatic polyurethanes, siloxane based aromatic polyurethanes, polydimethylsiloxane and other silicone rubbers, polyester, polyolefins, polymethyl-methacrylate, vinyl halide polymer and copolymers, polyvinyl aromatics, polyvinyl esters, polyamides, polyimides, polyethers and many others that can be dissolved in appropriate solvents and electrically spun on the stent.

Biodegradable fiber-forming polymers that can be used include, without limitation poly (L-lactic acid), poly (lactide-co-glycolide), polycaprolactone, polyphosphate ester, poly (hydroxy-butyrate), poly (glycolic acid), poly (DL-lactic acid), poly (amino acid), cyanocrylate, some copolymers and biomolecules such as DNA, silk, chitozan, collagen and cellulose.

These hydrophilic and hydrophobic polymers which are readily degraded by microorganisms and enzymes are suitable for encapsulating material for medicaments. In particular, polycaprolacton has a slower degradation rate than most other polymers and is therefore especially suitable for controlled-release of pharmaceutical agent over long periods of time scale ranging from about 2 years to about 3 years.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1. A medical device for implantation in a vessel, comprising at least one anastomotic member at least partially interposing a non-woven liner of electrospun fibers and a non-woven cover of electrospun fibers;
said at least one anastomotic member being designed for engaging at least one end of the medical device to a wall of the vessel upon implantation of the medical device within the vessel.

2. The device of claim 1, wherein said at least one anastomotic member protrudes out of said non-woven cover.

3. The device of claim 1, wherein said at least one anastomotic member is flush with said non-woven cover.

4-5. (canceled)

6. The device of claim 1, wherein said at least one anastomotic member comprises a plurality of hooks for connecting the device to said wall of the vessel.

7. The device of claim 1, further comprising a ring-shaped protuberance designed and constructed to prevent the device from falling into a lumen of the vessel.

8-14. (canceled)

15. The device of claim 1, forming a furcating structure having a plurality of tubular branches.

16-26. (canceled)

27. The device of claim 1, further comprising at least one adhesive layer for adhering at least two of: said non-woven liner, said non-woven cover and said at least one anastomotic member.

28-30. (canceled)

31. The device of claim 1, wherein said electrospun fibers of said non-woven liner and/or said non-woven cover are aligned at a predetermined orientation relative to a longitudinal axis of the device.

32. The device of claim 1, wherein said electrospun fibers of said non-woven liner and/or said non-woven cover are randomly aligned.

33. The device of claim 1, wherein at least a portion of said electrospun fibers of said non-woven liner and/or said non-woven cover are aligned substantially along a circumferential direction of said non-woven liner and/or said non-woven cover.

34. The device of claim 1, wherein at least one of said non-woven liner and said non-woven cover comprises at least one medicament incorporated therein, for delivery of said at least one medicament into a body vasculature during or after implantation of the device within said body vasculature.

35-39. (canceled)

40. A method of manufacturing a medical device for implantation in a vessel, the method comprising:
providing at least one anastomotic member designed for engaging a wall of the vessel;
electrospinning a first liquefied polymer on a precipitation electrode, thereby providing a non-woven liner of electrospun fibers;
mounting said at least one anastomotic member onto said precipitation electrode; and
electrospinning a second liquefied polymer on at least one of: said precipitation electrode, said non-woven liner and said at least one anastomotic member, so as to provide a non-woven cover of electrospun fibers.

41. The method of claim 40, wherein said electrospinning said second liquefied polymer is done such that said at least one anastomotic member protrudes out of said non-woven cover.

42. The method of claim 40, wherein said electrospinning said second liquefied polymer is done such that said at least one anastomotic member is flush with said non-woven cover.

43-46. (canceled)

47. The method of claim 41, further comprising mounting a thrust ring onto said non-woven cover, wherein said thrust ring is designed and constructed for thrusting said pressing ring.

48. (canceled)

49. The method of claim 40, further comprising repeating said electrospinning of said first and said second liquefied polymers for different orientations of said precipitation electrode, so as to form a furcating structure having a plurality of tubular branches.

50-60. (canceled)

61. The method of claim 40, further comprising applying pressure on at least one of said non-woven liner, said non-woven cover and said at least one anastomotic member.

62. The method of claim 40, further comprising electrospinning a third liquefied polymer prior to said mounting of said anastomotic member, wherein a boiling point of said third liquefied polymer is higher than a boiling point of said first liquefied polymer.

63. The method of claim 40, further comprising electrospinning a fourth liquefied polymer prior to said electrospinning of said second liquefied polymer, wherein a boiling point of said fourth liquefied polymer is higher than a boiling point of said second liquefied polymer.

64. The method of claim 40, further comprising applying at least one adhesive layer on at least one of said non-woven liner and said at least one anastomotic member.

65-72. (canceled)

73. The method of claim 40, wherein said electrospun fibers of said non-woven liner and/or said non-woven cover are aligned at a predetermined orientation relative to a longitudinal axis of the device.

74. The method of claim 40, wherein said electrospun fibers of said non-woven liner and/or said non-woven cover are randomly aligned.

75. The method of claim 40, wherein at least a portion of said electrospun fibers of said non-woven liner and/or said non-woven cover are aligned substantially along a circumferential direction of said non-woven liner and/or said non-woven cover.

76. The method of claim 40, wherein at least one of said non-woven liner and said non-woven cover comprises at least one medicament incorporated therein, for delivery of said at least one medicament into a body vasculature during or after implantation of the device within said body vasculature.

77-83. (canceled)

84. The method of claim 40, further comprising electrospinning an additional liquefied polymer on at least one of: said precipitation electrode, said non-woven liner, said at least one anastomotic member and said non-woven liner.

85-114. (canceled)

115. A kit for performing an end-to-side anastomosis procedure, comprising:
a medical device for implantation in a vessel, said medical device comprising at least one anastomotic member at least partially interposing a non-woven liner of electrospun fibers and a non-woven cover of electrospun fibers, wherein said at least one anastomotic member is designed for engaging at least one end of the medical device to a wall of said vessel upon implantation of said medical device within said vessel; and
an accessory device for forming an opening in said wall of said vessel, said accessory device comprising a tubular encapsulation designed and constructed for receiving said medical device, a cutting member integrated with or attached to an end of said tubular encapsulation and capable of forming an opening in said wall of said vessel, and a vacuum channel for channeling efflux of biological material from said tubular encapsulation.

116-119. (canceled)

120. The kit of claim 115, wherein said at least one anastomotic member comprises a plurality of hooks for connecting the device to said wall of the vessel.

121. The kit of claim 115, wherein said medical device further comprises a ring-shaped protuberance designed and constructed to prevent the device from falling into a lumen of the vessel.

122-157. (canceled)
